(19) United States
(12) Patent Application Publication
(10) Pub. No.: US 2005/0112658 A1
(43) Pub. Date: May 26, 2005

(54) Method of isolating and purifying a nucleic acid

(75) Inventor: Yoshihiko Makino, Asaka-shi (JP)

Correspondence Address: BIRCH STEWART KOLASCH & BIRCH, PO BOX 747, FALLS CHURCH, VA, 22040-0747, US

(73) Assignee: Fuji Photo Film Co., Ltd.

(21) Appl. No.: 10/975,469

(22) Filed: Oct. 29, 2004

(30) Foreign Application Priority Data
Oct. 31, 2003 (JP) 2003-373024
Oct. 31, 2003 (JP) 2003-373111
Sep. 24, 2004 (JP) 2004-277933

(51) Int. Cl. C12Q 1/68; C07H 21/04

(52) U.S. Cl. 435/6; 536/25.4

(57) ABSTRACT

A method of isolating and purifying a nucleic acid, comprises the steps of: (1a) passing a sample solution containing a nucleic acid through a nucleic acid-adsorptive porous membrane to adsorb the nucleic acid to the nucleic acid-adsorptive porous membrane under a specific condition; (2a) passing a wash solution through the nucleic acid-adsorptive porous membrane to wash the nucleic acid-adsorptive porous membrane while adsorbing the nucleic acid under a specific condition; and (3a) passing a elution solution through the nucleic acid-adsorptive porous membrane to desorb the nucleic acid from the nucleic acid-adsorptive porous membrane under a specific condition.

### TECHNICAL FIELD

The present invention relates to a method for isolation of nucleic acid from a sample solution containing the acid and its purification under reduced pressure or centrifugal force using a nucleic acid-adsorptive porous membrane.

### BACKGROUND ART

Various forms of nucleic acid are used in a variety of fields. For example, in the field of recombinant nucleic acid technology, nucleic acid is used in the form of probe, genomic nucleic acid and plasmid nucleic acid.

In the field of diagnostics, nucleic acid is used in various methods. For example, a nucleic acid probe is routinely used in the detection and diagnosis of a human pathogen. Likewise, it is used for the detection of genetic disorders. It is also used for the detection of a food contaminant. Moreover, it is routinely used in locating, identifying and isolating nucleic acid of interest for a variety of reasons ranging from genetic mapping to cloning and recombinant expression.

In many cases, nucleic acid is available in extremely small amounts. Moreover, its isolation/purification needs a time-consuming, sophisticated procedure, which tends to lead nucleic acid loss. Purification of nucleic acid isolated from a serum, urine or bacteria culture involves other problems, risks of contamination and yielding pseudo-positive results. One of the well-known isolation/purification methods comprises adsorption of nucleic acid on a solid phase, e.g., silicon dioxide, silica polymer or magnesium silicate, and subsequent step, e.g., washing or desorption (disclosed by, e.g., Patent Document 1: JP-B 7-51065). However, it involves problems of being not sufficient in simplicity, swiftness, and suitability for automation and reducing system size, although high in isolation performance. Its other problems come from its adsorbent, including difficulty in production of an adsorbent of identical performance on an industrial scale, handling and realizing various shapes.

One of the methods for isolation/purification of nucleic acids imply and efficiently uses a solution for adsorbing nucleic acid on a solid phase and another solution for desorbing it, wherein the solid phase is composed of an organic polymer having hydroxyl group on the surface (disclosed by, e.g., Patent Document 2: JP-A-2003-128691). This method, however, needs further improvement.

### DISCLOSURE OF THE INVENTION

It is an object of the present invention to provide a method of isolating and purifying a nucleic acid, comprising adsorption of nucleic acid in a sample on a nucleic acid-adsorptive porous membrane, and desorption of the acid by an adequate procedure, e.g., washing. It is another object of the present invention to provide a method of isolating and purifying a nucleic acid, which is excellent in isolation capability and good washing efficiency, can be simply and swiftly handled, is highly suitable for automation and reducing size of the system in which it is used, and can be mass-produced with substantially identical isolating capability.

The inventors of the present invention have found, after having extensively studied to solve the above problems, that nucleic acid can be isolated in a high yield from a sample containing the acid and purified to a high purity by a method comprising adsorption of the acid on a porous membrane and subsequent desorption of the acid, wherein the solution is passed through a nucleic acid-adsorptive porous membrane under reduced pressure or centrifugal force using a nucleic acid-adsorptive porous membrane, achieving the present invention based on these findings.

The present invention includes the following aspects.

1. A method of isolating and purifying a nucleic acid, comprising the steps of:
(1a) passing a sample solution containing a nucleic acid through a nucleic acid-adsorptive porous membrane to adsorb the nucleic acid to the nucleic acid-adsorptive porous membrane;
(2a) passing a wash solution through the nucleic acid-adsorptive porous membrane to wash the nucleic acid-adsorptive porous membrane while adsorbing the nucleic acid; and
(3a) passing a elution solution through the nucleic acid-adsorptive porous membrane to desorb the nucleic acid from the nucleic acid-adsorptive porous membrane,
wherein each of the sample solution, the wash solution and the elution solution in each of the steps (1a), (2a) and (3a) is passed through the nucleic acid-adsorptive porous membrane under a centrifugal force.

2. The method of isolating and purifying a nucleic acid according to the item 1, which comprising using a cartridge for isolation and purification of a nucleic acid, the cartridge comprising: a container provided with at least two openings; and the nucleic-acid-adsorptive porous membrane provided-in the container,
wherein each of the sample solution, the wash solution and the elution solution in each of the steps (1a), (2a) and (3a) is injected into the cartridge via one of the at least two openings of the container, is passed through the nucleic acid-adsorptive porous membrane by centrifuging the cartridge, and is discharged from the other opening of the container.

3. A method of isolating and purifying a nucleic acid, comprising the steps of:
(1b) passing a sample solution containing a nucleic acid through a nucleic acid-adsorptive porous membrane to adsorb the nucleic acid to the nucleic acid-adsorptive porous membrane;
(2b) passing a wash solution through the nucleic acid-adsorptive porous membrane to wash the nucleic acid-adsorptive porous membrane while adsorbing the nucleic acid; and
(3b) passing a elution solution through the nucleic acid-adsorptive porous membrane to desorb the nucleic acid from the nucleic acid-adsorptive porous membrane,
wherein each of the sample solution and the wash solution in each of the step (1b) or (2b) is passed through the nucleic acid-adsorptive porous membrane under reduced pressure, and the elution solution in the step (3b) is passed through the nucleic acid-adsorptive porous membrane under reduced-pressure or centrifugal force.

4. The method of isolating and purifying a nucleic acid according to the item 3, which comprising using a cartridge for isolation and purification of a nucleic acid, the cartridge comprising: a container provided with at least two openings; and the nucleic acid-adsorptive porous membrane provided in the container,
wherein each of the sample solution and the wash solution in each of the step (1b) or (2b) is injected into the cartridge via one of the at least two of openings of the container, is passed through the cartridge under reduced pressure generated by a differential pressure generator connected to the other opening of the container, and is discharged from the other opening of the container, and
the elution solution in the step (3b) is injected into the cartridge via one of the at least two of openings of the container, is passed through the cartridge under reduced pressure generated by a differential pressure generator connected to the other opening of the container or under a centrifugal force, and is discharged from the other opening of the container.

5. The method of isolating and purifying a nucleic acid according to any one of the items 1 to 4, wherein the nucleic acid-adsorptive porous membrane is a porous membrane-capable of adsorbing a nucleic acid by an interaction involving substantially no ionic bond.

6. The method of isolating and purifying a nucleic acid according to the item 5, wherein the porous nucleic acid-adsorptive membrane is a porous membrane comprising an organic polymer having a polysaccharide structure.

7. The method of isolating and purifying a nucleic acid according to the item 6, wherein the porous membrane comprising an organic polymer having a polysaccharide structure is a porous membrane comprising a mixture of acetyl cellulose having a different acetyl value.

8. The method of isolating and purifying a nucleic acid according to the item 7, wherein the mixture of acetyl cellulose having a different acetyl value is a mixture of triacetyl cellulose and diacetyl cellulose.

9. The method of isolating and purifying a nucleic acid according to the item 8, wherein the mixture contains triacetyl cellulose and diacetyl cellulose in a ratio of 99/1 to 1/99 by weight.

10. The method of isolating and purifying a nucleic acid according to the item 7, wherein the mixture of acetyl cellulose having a different acetyl value is a mixture of triacetyl cellulose and monoacetyl cellulose.

11. The method of isolating and purifying a nucleic acid according to the item 7, wherein the mixture of acetyl cellulose having a different acetyl value is a mixture of triacetyl cellulose, diacetyl cellulose and monoacetyl cellulose.

12. The method of isolating and purifying a nucleic acid according to the item 7, wherein the mixture of acetyl cellulose having a different acetyl value is a mixture of diacetyl cellulose and monoacetyl cellulose.

13. The method of isolating and purifying a nucleic acid according to the item 6, wherein the porous membrane comprising an organic polymer having a polysaccharide structure is a porous membrane comprising an organic material containing saponified acetyl cellulose.

14. The method of isolating and purifying a nucleic acid according to the item 13, wherein a saponification degree of the saponified acetyl cellulose is 5% or more.

15. The method of isolating and purifying a nucleic acid according to the item 13, wherein the porous membrane comprising an organic material containing saponified acetyl cellulose is a porous membrane comprising an organic material containing a saponified mixture of acetyl cellulose having a different acetyl value.

16. The method of isolating and purifying a nucleic acid according to the item 15, wherein a saponification degree of the saponified mixture of acetyl cellulose having a different acetyl value is 5% or more.

17. The method of isolating and purifying a nucleic acid according to the item 16, wherein the organic material containing the saponified mixture of acetyl cellulose having a different acetyl value is a saponified mixture of triacetyl cellulose and diacetyl cellulose.

18. The method of isolating and purifying a nucleic acid according to the item 17, wherein the ratio of triacetyl cellulose and diacetyl cellulose in the saponified mixture is 99/1 to 1/99 by weight.

19. The method of isolating and purifying a nucleic acid according to the item 16, wherein the organic material containing the saponified mixture of acetyl cellulose having a different acetyl value is a saponified mixture of triacetyl cellulose and monoacetyl cellulose.

20. The method of isolating and purifying a nucleic acid according to the item 16, wherein the organic material containing the saponified mixture of acetyl cellulose having a different acetyl value is a saponified mixture of triacetyl cellulose, diacetyl cellulose and monoacetyl cellulose.

21. The method of isolating and purifying a nucleic acid according to the item 16, wherein the organic material containing the saponified mixture of acetyl cellulose having a different acetyl value is a saponified mixture of diacetyl cellulose and monoacetyl cellulose.

22. The method of isolating and purifying a nucleic acid according to the item 13, wherein the nucleic acid-adsorptive porous membrane after saponification treatment has an average pore size smaller than that before the saponification treatment.

23. The method of isolating and purifying a nucleic acid according to the item 22, wherein the raito of an average pore size of the nucleic acid-adsorptive porous membrane after saponification treatment to that before the saponification treatment is 0.8 or less.

24. The method of isolating and purifying a nucleic acid according to the item 6, wherein the nucleic acid-adsorptive porous membrane is a porous membrane containing a regenerated cellulose.

25. The method of isolating and purifying a nucleic acid according to the item 5, wherein the nucleic acid-adsorptive porous membrane is a porous membrane obtained by treating a porous membrane comprising an organic material free of a hydrophilic group to introduce a hydrophilic group.

26. The method of isolating and purifying a nucleic acid according to the item 25, wherein the introduction of hydrophilic group to the porous membrane is carried out by binding a graft polymer chain having a hydrophilic group to the porous membrane.

27. The method of isolating and purifying a nucleic acid according to the item 5, wherein the nucleic acid-adsorptive porous membrane is a porous membrane obtained by coating an organic material free of a hydrophilic group with a material having a hydrophilic group to introduce a hydrophilic group.

28. The method of isolating and purifying a nucleic acid according to the item 27, wherein the material having a hydrophilic group is an organic polymer having a hydrophilic group.

29. The method of isolating and purifying a nucleic acid according to the item 5, wherein the nucleic acid-adsorptive porous membrane is a porous membrane comprising an inorganic material having a hydrophilic group in itself.

30. The method of isolating and purifying a nucleic acid according to the item 5, wherein the nucleic acid-adsorptive porous membrane is a porous membrane obtained by treating a porous membrane comprising an inorganic material free of a hydrophilic group to introduce a hydrophilic group.

31. The method of isolating and purifying a nucleic acid according to the item 30, wherein the introduction of a hydrophilic group to the porous membrane is carried out by binding a graft polymer chain having a hydrophilic group to the porous membrane.

32. The method of isolating and purifying a nucleic acid according to the item 5, wherein the nucleic acid-adsorptive porous membrane is a porous membrane obtained by coating a porous membrane comprising an inorganic material free of hydrophilic group with a material having a hydrophilic group to introduce a hydrophilic group.

33. The method of isolating and purifying a nucleic acid according to the item 32, wherein the material having a hydrophilic group is an organic polymer having a hydrophilic group.

34. The method of isolating and purifying a nucleic acid according to any one of the items 25, 27, 29, 30 and 32, wherein the hydrophilic group is a hydroxyl group.

35. The method of isolating and purifying a nucleic acid according to the item 5, wherein the nucleic acid-adsorptive porous membrane is a porous membrane, in which the front and back sides of the porous membrane are asymmetrical.

36. A cartridge for isolation and purification of a nucleic acid, comprising a nucleic acid-adsorptive porous membrane and a container provided with at least two openings, the cartridge being used for carrying out the method of isolating and purifying a nucleic acid according to any one of the items 1 to 35.

37. A kit comprising: a cartridge for isolation and purification of a nucleic acid; and a reagent, the kit being used for carrying out the method of isolating and purifying a nucleic acid according to any one of the items 1 to 35.

The method of the present invention for isolation/purification of nucleic acid, which passes a sample solution containing nucleic acid, wash solution and elution solution through a nucleic acid-adsorptive porous membrane under reduced pressure or centrifugal force, can isolate the nucleic acid from the sample solution and purify it at a high isolation efficiency, simply and swiftly in an automatic manner.

### BRIEF DESCRIPTION OF THE DRAWINGS

FIG. 1 presents a photograph showing DNA isolated/purified in EXAMPLE 1 by the method of the present invention and in COMPARATIVE EXAMPLE 1, and a molecular weight marker, obtained by electrophoresis.

### BEST MODE FOR CARRYING OUT THE INVENTION

One embodiment of the method of the present invention for isolation/purification of nucleic acid at least comprises the following steps, (1a) a step of passing a sample solution containing nucleic acid through a nucleic acid-adsorptive porous membrane under a centrifugal force to hold the acid within the membrane by adsorption, (2a) a step of passing a wash solution through the nucleic acid-adsorptive porous membrane under a centrifugal force to wash the membrane while it is adsorbing the acid, and (3a) a step of passing a elution solution through the nucleic acid-adsorptive porous membrane under a centrifugal force to desorb the acid from the membrane.

In the above embodiment, it is preferable that a cartridge for isolation and purification of a nucleic acid is used to hold the nucleic acid-adsorptive porous membrane in a container provided with at least two openings, and each of the sample solution, wash solution and elution solution is injected into the cartridge via one of the openings (hereinafter referred to as the inlet port), passed through the cartridge under a centrifugal force and discharged from the other opening (hereinafter referred to as the discharge port) in the step (1a), (2a) or (3a).

Passing the sample solution containing nucleic acid, wash solution and elution solution through the nucleic acid-adsorptive porous membrane under a centrifugal force reduces process time from injection of the sample solution to recovery of the nucleic acid from the cartridge, and hence is preferable.

In each of the steps (1a), (2a) and (3a), the centrifugal force is applied preferably at 5000 to 12000 rpm for 0.2 to 5 minutes, more preferably 7000 to 10000 rpm for 0.5 to 2 minutes, still more preferably 8000 for 1 minute.

Any centrifuge commonly used for generating a centrifugal force may be useful for the present invention. A high-speed centrifuge is more preferable. Each of the solutions is passed through the nucleic acid-adsorptive porous membrane under a centrifugal force, generated by a centrifuge set to direct the force towards the spent solution container which holds the spent sample solution and wash solution, or elution solution container which holds the spent elution solution, both described later, provided at the discharge port of the nucleic acid isolation/purification cartridge.

Another embodiment of the method of the present invention for isolation/purification of nucleic acid at least comprises the following steps, (1b) a step of passing a sample solution containing nucleic acid through a nucleic acid-adsorptive porous membrane under reduced pressure to hold the acid within the membrane by adsorption, (2b) a step of passing a wash solution through the nucleic acid-adsorptive porous membrane under reduced pressure to wash the membrane while it is adsorbing the acid, and (3b) a step of passing a elution solution through the nucleic acid-adsorptive porous membrane under reduced pressure or centrifugal force to desorb the acid from the membrane.

In the above embodiment, it is preferable that a cartridge for isolation and purification of a nucleic acid is used to hold the nucleic acid-adsorptive porous membrane in a container provided with at least two openings, and each of the sample solution and wash solution is injected into the cartridge via one of the openings (inlet port), passed through the cartridge under reduced pressure generated by a differential pressure generator connected to the other opening (discharge port) and discharged from the discharge port in the step (1b) or (2b), whereas the elution solution is injected into the cartridge via the inlet port, passed through the cartridge under reduced pressure generated by a differential pressure generator connected to the discharge port or under a centrifugal force and discharged from the discharge port in the step (3b). Passing the sample solution containing nucleic acid and wash solution through the nucleic acid-adsorptive porous membrane under reduced pressure, while passing the elution solution through the membrane under reduced pressure or centrifugal force reduces process time from injection of the sample solution to recovery of the nucleic acid from the cartridge, and hence is preferable.

In each of the steps (1b), (2b) and (3b), each solution is passed through the membrane under a pressure of preferably around −10 to −80 kPa, more preferably −30 to −60 kPa, when it is kept under reduced pressure. The differential pressure generator may be a syringe, evaporator, aspirator, vacuum pump, or vacuum pump which may be connected to a vacuum chamber or the like to keep the membrane under reduced pressure. Of these, a syringe is preferable for a manual operation, whereas a vacuum pump is preferable for an automatic operation.

It is preferable to use a differential pressure generator, in which the degree of vacuum can reach the degree of reduced pressure described above.

The generator is preferably connected detachably to the discharge port of the nucleic acid isolation/purification cartridge.

In the step (3b), the centrifugal force is applied preferably at 5000 to 12000 rpm, more preferably 7000 to 10000 rpm. It is preferably kept for 0.5 to 1.5 minutes, more preferably 1 minute.

Any centrifuge commonly used for generating a centrifugal force may be useful for the present invention. A high-speed centrifuge is more preferable. The elution solution is passed through the nucleic acid-adsorptive porous membrane under a centrifugal force generated by a centrifuge, after the elution solution container which holds the elution solution, described later, is set at the discharge port of the nucleic acid isolation/purification cartridge.

The nucleic acid isolation/purification step can recover nucleic acid whose molecular weight varies over a wide range from 1 to 200 kbp, in particular 20 to 140 kbp. In other words, it can recover nucleic acid of longer chain than the conventional spin column method which uses a glass filter.

Moreover, the nucleic acid isolation/purification step can steadily recover highly pure nucleic acid containing a limited quantity of impurities, having a purity of 1.6 to 2.0 in the case of DNA and 1.8 to 2.2 in the case of RNA, determined by an ultraviolet-visible spectrophotometer (260/280 nm). Still more, it can recover nucleic acid having a purity around 1.8 in the case of DNA and around 2.0 in the case of the RNA, determined by an ultraviolet-visible spectrophotometer (260/280 nm).

There is not limit to the sample for the present invention. In the field of diagnostics, for example, the samples to which the present invention include collected body fluids, e.g., whole blood, plasma, serum, urine, stool, sperm and saliva, plants (or a part thereof), animals (or a part thereof), and solutions prepared from biological materials, e.g., those of bacteria, viruses, cultured cells, and lysates and homogenates thereof.

First, these samples are treated with an aqueous solution containing a reagent which dissolves cell membranes and solubilizes nucleic acid (nucleic acid solubilizing reagent) This allows cell membranes and nuclear membranes to be dissolved, and nucleic acid to be dispersed in the aqueous solution, to prepare the sample solution containing nucleic acid.

For dissolving cell membranes and nucleic membrane to solubilize nucleic acid, for example, when a sample is whole blood, (A) removal of erythrocytes, (B) removal of various proteins, and (C) lysis of leukocytes and nuclear membranes are necessary. (A) Removal of erythrocytes and (B) removal of various proteins are necessary to prevent their non-specific adsorption on and clogging of the porous membrane, and (C) lysis of leukocytes and nuclear membranes is necessary to solubilize nucleic acid which is to be extracted. In particular, (C) lysis of leukocytes and nuclear membranes is an essential step to solubilize nucleic acid.

The sample solution for the present invention may contain single type of nucleic acid or 2 or more different types of nucleic acid. The nucleic acid type to be recovered is not limited. It may be DNA, RNA, single-stranded, double-stranded, linear or cyclic. Number of samples is not limited. It may be 1 or more. For example, 2 or more samples may be processed simultaneously by a parallel system provided with a corresponding number of containers. Length of nucleic acid to be recovered is also not limited. It may be optional in a range from several bp to several Mbp. It is however generally in a range from several bp to several hundreds bp for ease of handling. The nucleic acid isolation/purification method of the present invention can swiftly recover longer nucleic acid than a conventional simple method. Length of nucleic acid to be recovered by the present invention is preferably 50 kbp or longer, more preferably 100 kbp or longer, still more preferably 100 kbp or longer.

Preparation of a sample solution containing nucleic acid from a sample is described. This step dissolves cell and nuclear membranes to solubilize nucleic acid in the presence-of a nucleic acid solubilizing reagent, e.g., a chaotropic salt, surfactant or protease solution.

One example of this step for preparing a sample solution containing nucleic acid from a sample by dissolving cell and nuclear membranes to solubilize nucleic acid comprises the following sub-steps:
(I) Injection of a sample into a container,
(II) Mixing the sample with a nucleic acid solubilizing reagent containing a chaotropic salt and surfactant, incorporated in the container,
(III) Incubation of the resulting mixture, and
(IV) Incorporation of a water-soluble, organic solvent in the incubated mixture

The above step, which prepares a sample solution containing nucleic acid from a sample by dissolving cell and nuclear membranes to solubilize nucleic acid, will have improved suitability for automatic treatment, when the sample is homogenized. This treatment may be carried out with the aid of ultrasonic waves, sharp projections, high-speed agitation, extrusion through fine voids or glass beads.

Moreover, the above step, which prepares a sample solution containing nucleic acid from a sample by dissolving cell and nuclear membranes to solubilize nucleic acid, will have improved nucleic acid recovery yield and efficiency, leading to reduced requirement for the sample containing nucleic acid and speeding-up the step process, when a protease is incorporated in the nucleic acid solubilizing reagent.

The preferable protease for the present invention is at least one selected from, e.g., serine, cystine and metallic protease. A mixture of 2 or more proteases is also preferable.

Serine protease type is not limited, and the preferable ones include protease K.

Cystine protease type is also not limited, and the preferable ones include papain and cathepsin.

Metallic protease type is also not limited, and the preferable ones include carboxypeptidase.

A protease is incorporated preferably at 0.0011 to 10 IU per 1 mL of the total reaction system volume, more preferably 0.01 to 1 IU.

Moreover, a protease free of nuclease is preferably used. Still more, a protease containing a stabilizer is preferably used. A metallic ion is preferably used as a stabilizer. More specifically, the magnesium ion in the form of magnesium chloride or the like, is preferable. Incorporation of a stabilizer reduces required quantity of protease, thus reducing the required cost for nucleic acid recovery. A stabilizer is incorporated preferably at 1 to 1000 mmols per 1 mL of the total reaction system volume, more preferably 10 to 100 mmols/L.

A protease may be incorporated beforehand with a chaotropic salt, surfactant or another reagent to be used as a mixed reagent for recovery of nucleic acid.

A protease and one or more other reagents may be used individually. In this case, a protease may be first incorporated in a sample and is then mixed with a reagent containing chaotropic salt, surfactant or the like. This order may be reversed, i.e., a reagent containing chaotropic salt, surfactant or the like is incorporated first and then mixed with a protease.

A protease may be directly incorporated dropwise, like an eye lotion, from a protease container in a mixture of sample and mixed reagent containing a chaotropic salt and and/or surfactant. This will simplify the mixing procedure.

A nucleic acid solubilizing reagent is incorporated preferably while being kept dried. A container holding a protease dried beforehand by freeze-drying or the like may be used. A sample solution containing nucleic acid may be prepared using 2 containers, one holding a dried nucleic acid solubilizing reagent and the other dried protease.

The above procedure can simply prepare a sample solution containing nucleic acid without changing nucleic acid yield, because the nucleic acid solubilizing reagent and protease are preserved well stably.

The procedure for mixing a sample with a nucleic acid solubilizing reagent containing a chaotropic salt and and/or surfactant is not-limited.

They are preferably mixed with each other by an agitator at 30 to 3000 rpm for 1 second to 3 minutes. This will increase an isolated/purified nucleic acid yield. Mixing with inversion is carried out preferably 10 to 50 times. Moreover, they may be mixed by pipetting carried out 10 to 50 times. This will increase an isolated/purified nucleic acid yield by the simple procedure.

The isolated/purified nucleic acid can be recovered in an increased yield, when a mixed solution of sample and nucleic acid solubilizing reagent containing a chaotropic salt and/or surfactant is incubated under suitable conditions with respect to reaction temperature and time for the protease. Incubation temperature is normally in a range from 20 to 70° C., preferably a suitable temperature for the protease. Incubation time is normally in a range from 1 to 90 minutes, preferably a suitable time for the protease. The incubation procedure is not limited. It may be carried out using a water bath or heater.

In the above step, which prepares a sample solution containing nucleic acid from a sample by dissolving cell and nuclear membranes to solubilize nucleic acid, the nucleic acid solubilizing reagent containing a chaotropic salt and surfactant is kept preferably at a pH of 5 to 10, more preferably 6 to 9, still more preferably 7 to 8.

Moreover, in the above step, which prepares a sample solution containing nucleic acid from a sample by dissolving cell and nuclear membranes to solubilize nucleic acid, the chaotropic salt is incorporated in the nucleic acid solubilizing reagent preferably at 0.5 mols/L or more, more preferably 0.5 to 4 mols/L, still more preferably 1 to 3 mols/L. The chaotropic salt is preferably of guanidine hydrochloride, although another chaotropic salt (e.g., urea, sodium iodide, potassium iodide, guanidine isothiocyanate or thiocyanate) may be used. These salts may be used either individually or in combination.

The nucleic acid solubilizing reagent may contain a water-soluble, organic solvent, to improve solubility of the compound contained in the reagent. The water-soluble, organic solvent is preferably of alcohol. It may be primary, secondary or tertiary. The preferable ones include methanol, ethanol, propanol and an isomer thereof, and butanol and an isomer thereof. They may be used either individually or in combination. It is incorporated in the nucleic acid solubilizing reagent preferably at 1 to 20% by weight.

In the above step, which prepares a sample solution containing nucleic acid from a sample by dissolving cell and nuclear membranes to solubilize nucleic acid, the surfactant to be incorporated may be nonionic, cationic, anionic or amphoteric.

A nonionic surfactant is more preferable for the present invention. The nonionic surfactants useful for the present invention include those based on polyoxyethylene alkyl, phenyl ether, polyoxyethylene alkyl ether and fatty acid alkanol amide, of which those based on polyoxyethylene alkyl phenyl ether and polyoxyethylene alkyl ether are more preferable. Of those based on polyoxyethylene alkyl phenyl ether, POE octyl phenyl ether is more preferable. Of those based on polyoxyethylene alkyl ether, the more preferable ones include those selected from the group consisting of POE decyl ether, POE lauryl ether, POE tridecyl ether, POE alkylene decyl ether, POE sorbitan monolaurate, POE sorbitan monooleate, POE sorbitan monostearate, polyoxyethylene sorbit tetraoleate, POE alkyl amine and POE acetylene glycol.

A cationic surfactant is also preferably used. More preferable cationic surfactants include those selected from the group consisting of cetyltrimethylammonium bromide, dodecyltrimethylammonium chloride, tetradecyltrimethylammonium chloride and cetylpyridinium chloride. They may be used either individually or in combination. The surfactant is incorporated in the nucleic acid solubilizing reagent preferably at 1 to 20% by weight.

In the above step, which prepares a sample solution containing nucleic acid from a sample by dissolving cell and nuclear membranes to solubilize nucleic acid, the nucleic acid solubilizing reagent is preferably incorporated with a ribonuclease, when nucleic acid other than DNA or RNA is to be recovered. This can reduce interference by RNA present in the recovered nucleic acid. Incorporation of a deoxyribonuclease inhibitor is also preferable.

Nucleic acid, e.g., RNA, other than DNA is to be recovered, on the other hand, the nucleic acid solubilizing reagent is preferably incorporated with a deoxyribonuclease. This can reduce interference by DNA present in the recovered nucleic acid. Incorporation of a ribonuclease inhibitor is also preferable. It is preferable that the ribonuclease inhibitor specifically inhibits the ribonuclease.

Ribonuclease is not limited for the present invention. The preferable ones include a specific one, e.g., ribonuclease RNase H.

Deoxyribonuclease is not limited for the present invention. The preferable ones include a specific one, e.g., deoxyribonuclease DNase I.

A nuclease and its inhibitor may be incorporated at a concentration normally used. They may be heated by a common procedure. The heat treatment is preferably carried out simultaneously with the treatment with a protease.

In the above step, which prepares a sample solution containing nucleic acid from a sample by dissolving cell and nuclear membranes to solubilize nucleic acid, the sample solution containing nucleic acid is also preferably incorporated with a defoaming agent. The preferable defoaming agents include a combination of two components of silicon- and alcohol-based ones. The preferable alcohol-based defoaming agents include a surfactant of acetylene glycol.

More specifically, the preferable defoaming agents include those based on silicon (e.g., silicone oil, dimethyl polysiloxane, silicone emulsion, modified polysiloxane and silicone compound), alcohol (e.g., acetylene glycol, heptanol, ethylhexanol, higher alcohol and polyoxyalkylene glycol), ether (e.g., heptyl cellsorb and nonyl cellsorb-3-heptylcorbitol), oil and fat (e.g., animal and vegetable oil), fatty acid (e.g., stearic, oleic and palmitic acid), metallic soap (aluminum and calcium stearate), fatty acid ester (natural wax and tributyl phosphate), phosphoric acid ester (e.g., sodium octylphosphate), amine (e.g., dimethylamine), and amide (e.g., amide stearate). The other preferable defoaming agents include ferric sulfate and bauxite. The particularly preferable defoaming agents include a combination of two components of silicon- and alcohol-based ones. The preferable alcohol-based defoaming agents include a surfactant of acetylene glycol.

In the step (IV) of incorporating a water-soluble, organic solvent in the incubated mixture solution as part of the above step, which prepares a sample solution containing nucleic acid from a sample by dissolving cell and nuclear membranes to solubilize nucleic acid, alcohol is a preferable water-soluble, organic solvent. It may be primary, secondary or tertiary. The preferable ones include methanol, ethanol, propanol, butanol and an isomer thereof. It is incorporated in the nucleic acid solubilizing reagent preferably at 5 to 90% by weight as the final concentration in the sample solution containing nucleic acid.

In the above step, which prepares a sample solution containing nucleic acid from a sample by dissolving cell and nuclear membranes to solubilize nucleic acid, the resulting sample solution containing nucleic acid preferably has a surface tension of 0.05 J/m² or less, viscosity of 1 to 10,000 mPas and specific gravity of 0.8 to 1.2.

In the method of isolating and purifying a nucleic acid in the present invention, the nucleic acid-adsorptive porous membrane is used. The porous membrane can be mass-produced with substantially identical isolating capability.

Next, the nucleic acid-adsorptive porous membrane and adsorption step for the present invention are described. The nucleic acid-adsorptive porous membrane for the present invention is the one which allows a solution to pass therethrough. A membrane “which allows a solution to pass therethrough” means that the solution can pass the membrane, when a centrifugal force is applied to the membrane, in the direction of the centrifugal force, or when a differential pressure is applied between a space with which the membrane comes into contact at one side and another space with which it comes into contact at the other side, in the direction from the higher-pressure side towards the lower-pressure side.

It is preferable for the nucleic acid-adsorptive porous membrane for the present invention to adsorb nucleic acid by interactions involving substantially no ionic bond, by which is meant that the porous membrane is not “ionized” under the service conditions, where the nucleic acid and porous membrane conceivably attract each other by changing polarity of the service atmosphere. This allows the porous membrane to exhibit high isolation performance and washing efficiency for isolation/purification of nucleic acid. It is more preferable for the nucleic acid-adsorptive porous membrane to have a hydrophilic group, where the nucleic acid and porous membrane conceivably attract each other by changing polarity of the service atmosphere. The porous membrane having a hydrophilic group means that the material constituting the membrane has a hydrophilic group, or treated or coated to have a hydrophilic group. The material constituting the porous membrane may be organic or inorganic. For example, the porous membrane may be made of an organic material originally having a hydrophilic group, organic material originally having no hydrophilic group but treated to have a hydrophilic group, organic material originally having no hydrophilic group but coated with a material having a hydrophilic group, inorganic material originally having a hydrophilic group, inorganic material originally having no hydrophilic group but treated to have a hydrophilic group, or inorganic material originally having no hydrophilic group but coated with a material having a hydrophilic group. However, the porous membrane is preferably made of an organic material, e.g., organic polymer, for ease of fabrication.

A hydrophilic group means a polar group (or radical) interactive with water. All of the groups (or radicals) involved in adsorption of nucleic acid are hydrophilic. Hydrophilic groups preferable for the present invention are those moderately interactive with water (“not highly hydrophilic groups,” according to ENCYCLOPAEDIA CHIMICA, Kyoritsu Shuppan). These include hydroxyl, carboxyl, cyano and oxyethylene, of which hydroxyl is more preferable.

Organic materials having a hydrophilic group for the porous membrane include polyhydroxyethyl acrylate, polyhydroxyethyl methacrylate, polyvinyl alcohol, polyvinyl pyrrolidone, polyacrylic acid, polymethacrylic acid, polyoxyethylene, acetyl cellulose and a mixture of acetyl cellulose compounds of different acetyl value, of which an organic polymer having a polysaccharide structure is more preferable.

Organic polymers having a polysaccharide structure include cellulose, hemicellulose, dextran, agarose, dextrin, amylose, amylopectin, starch, glycogen, pullulan, mannan, glucomannan, lichenan, isolichenan, laminaran, carrageenan, xylan, fluctan, alginic acid, hyaluronic acid, chondroitin, chitin and chitosan. Derivatives of these polysaccharide structures are also useful. The organic polymers for the present invention are not limited to above, so long as they are of polysaccharide structure or derivatives thereof. The derivatives include polysaccharide structures whose hydroxyl group is esterified, etherified or halogenated at an optional degree of substitution. The derivatives are more preferably saponified.

The esterified derivative of polysaccharide structure is preferably at least one selected from esters of carboxylic, nitric, sulfuric, sulfonic, phosphoric, phosphonic and pyrophosphoric acid. These esters of carboxylic, nitric, sulfuric, sulfonic, phosphoric, phosphonic and pyrophosphoric acid are more preferably saponified.

The carboxylic acid ester is preferably at least one selected from the group consisting of alkyl carbonyl, alkenyl carbonyl, aromatic carbonyl and aromatic alkyl carbonyl esters. These esters are more preferably saponified.

The alkyl carbonyl esters preferably has at least one group selected from the group consisting of acetyl, propionyl, butyloyl, valeric, heptanoyl, octanoyl, decanoyl, dodecanoyl, tridecanoyl, hexadecanoyl and octadecanoyl. These esters are more preferably saponified.

The alkenyl carbonyl esters preferably has at least one group selected from the group consisting of acrylic and methacrylic. These esters are more preferably saponified.

The aromatic carbonyl esters preferably has at least one group selected from the group consisting of benzoyl and naphthaloyl. These esters are more preferably saponified.

The preferable nitric acid esters include nitrocellulose, nitrohemicellulose, nitrodextran, nitroagarose, nitrodextrin, nitroamylose, nitroamylopectin, nitroglycogen, nitropullulan, nitromannan, nitroglucomannan, nitrolichenan, nitroisolichenan, nitrolaminaran, nitrocarrageenan, nitroxylan, nitrofluctan, nitroalginic acid, nitrohyaluronic acid, nitrochondroitin, nitrochitin and nitrochitosan. These esters are more preferably saponified. The preferable sulfuric acid esters include sulfates of cellulose, hemicellulose, dextran, agarose, dextrin, amylose, amylopectin, glycogen, pullulan, mannan, glucomannan, lichenan, isolichenan, laminaran, carrageenan, xylan, fluctan, alginic acid, hyaluronic acid, chondroitin, chitin and chitosan. These esters are more preferably saponified.

The sulfonic acid ester of polysaccharide structure is preferably at least one selected from the group consisting of alkyl sulfonic, alkenyl sulfonic, aromatic sulfonic and aromatic alkyl sulfonic esters. These esters are more preferably saponified.

The preferable phosphoric acid esters include phosphates of cellulose, hemicellulose, dextran, agarose, dextrin, amylose, amylopectin, glycogen, pullulan, mannan, glucomannan, lichenan, isolichenan, laminaran, carrageenan, xylan, fluctan, alginic acid, hyaluronic acid, chondroitin, chitin and chitosan. These esters are more preferably saponified.

The preferable phosphonic acid esters include phosphonates of cellulose, hemicellulose, dextran, agarose, dextrin, amylose, amylopectin, glycogen, pullulan, mannan, glucomannan, lichenan, isolichenan, laminaran, carrageenan, xylan, fluctan, alginic acid, hyaluronic acid, chondroitin, chitin and chitosan. These esters are more preferably saponified.

The preferable pyrophosphoric acid esters include pyrophosphates of cellulose, hemicellulose, dextran, agarose, dextrin, amylose, amylopectin, glycogen, pullulan, mannan, glucomannan, lichenan, isolichenan, laminaran, carrageenan, xylan, fluctan, alginic acid, hyaluronic acid, chondroitin, chitin and chitosan. These esters are more preferably saponified.

The preferable ether derivatives include methyl cellulose, ethyl cellulose, carboxymethyl cellulose, carboxyethyl cellulose, carboxyethyl-carbamoylethyl cellulose, hydroxymethyl cellulose, hydroxyethyl cellulose, hydroxypropyl cellulose, hydroxypropylmethyl cellulose, hydroxyethylmethyl cellulose, cyanoethyl cellulose and carbamoylethyl cellulose, although not limited thereto, of which hydroxymethyl cellulose and hydroxyethyl cellulose are more preferable.

The preferable materials for the porous membrane composed of an organic polymer having a polysaccharide structure include acetyl cellulose. A mixture of acetyl cellulose compounds of different acetyl value is also preferable for the membrane. The mixtures of acetyl cellulose compounds of different acetyl value include those of triacetyl cellulose and diacetyl cellulose, triacetyl cellulose and monoacetyl cellulose, triacetyl cellulose, diacetyl cellulose and monoacetyl cellulose, and diacetyl cellulose and monoacetyl cellulose.

Of these, a mixture of triacetyl cellulose and diacetyl cellulose is more preferable. The triacetyl cellulose/diacetyl cellulose ratio is preferably 99.1/1 to 1/99 by weight, more preferably 90/10 to 50/50.

The particularly preferable materials for the porous membrane composed of an organic polymer having a polysaccharide structure include saponified acetyl cellulose, e.g., a porous membrane of surface-saponified acetyl cellulose disclosed by JP-A 2003-128691. Surface-saponified acetyl cellulose means saponified acetyl cellulose or a mixture of acetyl cellulose compounds of different acetyl value. The preferable saponified mixtures of acetyl cellulose compounds include those of triacetyl cellulose and diacetyl cellulose, triacetyl cellulose and monoacetyl cellulose, triacetyl cellulose, diacetyl cellulose and monoacetyl cellulose, and diacetyl cellulose and monoacetyl cellulose, of which a mixture of triacetyl cellulose and diacetyl cellulose is more preferable. The triacetyl cellulose/diacetyl cellulose ratio is preferably 99.1/1 to 1/99 by weight, more preferably 90/10 to 50/50. Density of hydroxyl group on the porous membrane surface can be controlled by changing degree of saponification. Increasing hydroxyl group density increases nucleic acid isolation efficiency. Degree of saponification (degree of surface saponification) of the saponified porous membrane is preferably 5 to 100%, inclusive, more preferably 10 to 100%, also inclusive.

The nucleic acid-adsorptive porous membrane, when saponified, preferably has an average pore size smaller than that before the saponification treatment. More preferably, the saponified porous membrane has an average pore size of 0.8 times or less of that before the saponification treatment, still more preferably 0.5 times or less.

The saponification treatment is carried out by bringing acetyl cellulose with a saponification treatment solution (e.g., aqueous solution of sodium hydroxide). Part of acetyl cellulose coming into the solution is modified to have hydroxyl group. There generated cellulose has different characteristics, e.g., crystal condition, from those of the original one. The porous membrane for the present invention preferably contains the regenerated cellulose.

Degree of saponification can be controlled by changing concentration of sodium hydroxide. It can be easily determined by NMR, IR or XPS (for example, by reduced degree of carbonyl group peak).

One of the methods for introducing a hydrophilic group in the porous membrane of an organic material free of hydrophilic group is binding a graft polymer chain having a hydrophilic group to the membrane.

A graft polymer chain having a hydrophilic group can be bound to the porous membrane either via a chemical bond or by polymerization of a compound having a polymerizable double bond to produce the graft polymer chain on the porous membrane, which serves as a reaction starting point.

The method for binding a graft polymer chain to the porous membrane via a chemical bond uses a polymer having, at the main chain terminal or in the side chain, a functional group reactive with the porous membrane, where the functional group reacts with a functional group in the porous membrane for grafting. The functional group reactive with the porous membrane is not limited, so long as it is reactive with a functional group in the porous membrane. Some of these functional groups are a silane coupling group, e.g., alkoxysilane, and isocyanate, amino, hydroxyl, carboxyl, sulfonic acid, phosphoric acid, epoxy, allyl, methacryloyl and acryloyl groups.

The particularly useful polymers having a reactive, functional group at the main chain terminal or in the side chain include those having trialkoxysilyl, amino, carboxyl, epoxy or isocyanate group at the main chain terminal. These polymers are not limited, so long as they have a hydrophilic group which can accelerate adsorption of nucleic acid by the porous membrane. More specifically, they include polyhydroxyethylacrylic acid, polyhydroxyethylmethacrylic acid and a salt thereof; polyvinyl alcohol, polyvinyl pyrrolidone, polyacrylic acid, polymethacrylic acid and a salt thereof; and polyoxyethylene.

The method for polymerization of a compound having a polymerizable double bond to produce the graft polymer chain on the porous membrane serving as a reaction starting point is commonly referred to as surface graft polymerization. This polymerization method produces active species on the base material surface by plasma or light irradiation or heating, which activate polymerization of a compound having a polymerizable double bond, brought into contact with the porous membrane, to bind the graft polymer chain to the membrane. A compound for forming a graft polymer chain bound to a base material should have a polymerizable double bond and, at the same time, a hydrophilic group which can accelerate adsorption of nucleic acid. Such a compound may be a polymer, oligomer or monomer having a hydrophilic group, so long as it has a double bond in the molecular structure. A monomer having a hydrophilic group is particularly useful.

More specifically, the particularly useful monomers having a hydrophilic group are those having hydroxyl group, e.g., 2-hydroxyethylacrylate, 2-hydroxyethylmethacrylate and glycerol monomethacrylate. The other useful monomers include those having carboxyl group, e.g., acrylic acid, methacrylic acid and alkali metal or amine salt thereof.

Another method for introducing a hydrophilic group in the porous membrane of an organic material free of hydrophilic group is coating the membrane with a material having a hydrophilic group. The coating material is not limited, so long as it has a hydrophilic group which can accelerate adsorption of nucleic acid. However, an organic polymer is preferable for its high workability. The useful polymers include polyhydroxyethyl acrylate, polyhydroxyethyl methacrylate and a salt thereof; polyvinyl alcohol, polyvinyl pyrrolidone, polyacrylic acid, polymethacrylic acid and a salt thereof; polyoxyethylene, acetyl cellulose and a mixture of acetyl cellulose compounds of different acetyl value, of which an organic polymer having a polysaccharide structure is more preferable.

Acetyl cellulose and a mixture of acetyl cellulose compounds of different acetyl value may be saponified, after its covers the porous membrane of an organic material free of hydrophilic group. Degree of saponification is preferably 5% or more, more preferably 10% or more.

One example of the porous membrane of an inorganic material free of hydrophilic group is that contains a silica compound. A glass filter is one of the typical porous membranes of a silica compound. A porous, thin film of silica, disclosed by JP 3,058,342, is another example. It can be produced by following steps: a developing solution of a cationic, amphoteric material capable of forming a bilayer is developed over a base board to form a liquid film; the solvent is removed from the liquid film to form a thin, multilayered bilayer of the amphoteric material; the resulting bilayer is brought into contact with a solution containing a silica compound; and the bilayer is removed by extraction.

One of the methods for introducing a hydrophilic group in the porous membrane of an inorganic material free of hydrophilic group is to bind a graft polymer chain having a hydrophilic group to the membrane.

A graft polymer chain having a hydrophilic group can be bound to the porous membrane either via a chemical bond or by polymerization of a compound having a polymerizable double bond to produce the graft polymer chain on the porous membrane, which serves as a reaction starting point.

First, the method for binding a graft polymer chain to the porous membrane via a chemical bond uses a polymer having, at the main chain terminal or in the side chain, a functional group reactive with the porous membrane, where the functional group reacts with a functional group in the porous membrane for grafting.

In the above method, an inorganic material containing a functional group reactive with the functional group in the graft polymer is introduced, where these functional groups react with each other to chemically bind the graft polymer chain to the porous membrane. In the method for polymerization of a monomer having a polymerizable double bond and hydrophilic group in the molecular structure, on the other hand, a functional group to serve as a starting point for the polymerization is introduced into an inorganic material. The polymerization binds the resulting graft polymer chain to the porous membrane, which also serves as a reaction starting point. The graft polymer having a hydrophilic group and monomer having a double bond and hydrophilic group in the molecular structure are preferably the graft polymer having a hydrophilic group and monomer having a double bond and hydrophilic group in the molecular structure, respectively, described in the above method which chemically binds the graft polymer chain to the porous membrane of an organic material free of hydrophilic group. Another method for introducing a hydrophilic group in the porous membrane of an inorganic material free of hydrophilic group is coating the membrane with a material having a hydrophilic group. The coating material is not limited, so long as it has a hydrophilic group which can accelerate adsorption of nucleic acid. However, an organic polymer is preferable for its high workability. The useful polymers include polyhydroxyethyl acrylate, polyhydroxyethyl methacrylate and a salt thereof; polyvinyl alcohol, polyvinyl pyrrolidone, polyacrylic acid, polymethacrylic acid and a salt thereof; polyoxyethylene, acetyl cellulose and a mixture of acetyl cellulose compounds of different acetyl value.

Acetyl cellulose and a mixture of acetyl cellulose compounds of different acetyl value may be saponified, after its covers the porous membrane of an inorganic material free of hydrophilic group. Degree of saponification is preferably 5% or more, more preferably 10% or more.

The inorganic materials free of hydrophilic group which can be used for the porous membrane include metals, e.g., aluminum, glass, cement, ceramics, e.g., porcelain, new ceramics, silicon and activated coal.

The nucleic acid-adsorptive porous membrane may be 10 to 500 μm thick, more preferably 50 to 250 μm. The thinner, the better for ease of washing.

Moreover, the nucleic acid-adsorptive porous membrane may have an average pore size of 0.9 to 5.0 μm, more preferably 1.5 to 3.5 μm. This will secure a sufficient surface area for adsorbing nucleic acid while preventing clogging. The average pore size can be determined by the bubble point method (ASTM316-86, and JIS K-3832).

The nucleic acid-adsorptive porous membrane may be symmetrical with respect to the front and back sides, although preferably asymmetrical. The asymmetry means that the membrane has physical or chemical properties changing from one side to the other. The physical properties include average pore size, and chemical properties include degree of saponification, described earlier. It is preferable, when an asymmetrical membrane is used, that the pore size tapers off in the direction of liquid flow. The ratio of the largest pore size/smallest pore size ratio is preferably 2 or more for the porous membrane, more preferably 5 or more. This will secure a sufficient surface area for adsorbing nucleic acid while preventing clogging.

The nucleic acid-adsorptive porous membrane may have a void volume fraction of 50 to 95%, preferably 65 to 80%. Moreover, it may have a bubble point of 0.1 to 10 kgf/cm², preferably 0.2 to 4 kgf/cm².

The nucleic acid-adsorptive porous membrane preferably works at a pressure drop of 0.1 to 100 kPa. This will secure a uniform pressure drop across the membrane while it is pressurized, where pressure drop is defined as the minimum pressure required for passing water across a thickness of 100 μm. It is more preferably 0.5 to 50 kPa.

Moreover, the nucleic acid-adsorptive porous membrane may pass water at 1 to 500 mL/minute·cm² at 25° C. and a pressure of 1 kg/cm², preferably 5 to 1000 mL/minute·cm² at 25° C.

Still more, the nucleic acid-adsorptive porous membrane preferably adsorbs nucleic acid at 0.1 μg/mg or more, more preferably 0.9 μg/mg or more.

Still more, one of the preferable materials for the nucleic acid-adsorptive porous membrane is a cellulose derivative which is not dissolved within 1 hour in 5 mL of trifluoroacetic acid but dissolved within 48 hours, when a 5 mm square membrane is immersed. Another preferable material is a cellulose derivative which is dissolved within 1 hour in 5 mL of trifluoroacetic acid but is not dissolved within 24 hours in 5 mL of dichlorometane, when a 5 mm square membrane is immersed.

The nucleic acid-adsorptive porous membrane preferably passes a sample solution containing nucleic acid in one direction from one side to the other, to bring the solution uniformly in contact with the membrane, more preferably from the larger pore size side to the smaller pore size side to prevent clogging. Moreover, the nucleic acid-adsorptive porous membrane preferably passes a sample solution containing nucleic acid at 2 to 1500 L/second·cm² to secure contact time at an adequate level, more preferably 5 to 700 L/second·cm². A sufficient isolation/purification effect may not be secured when contact time is excessively short. On the other hand, system workability may deteriorate when contact time is excessively long.

Number of the nucleic acid-adsorptive porous membrane may be one or more. When two or more membranes are to be used, they may be the same or different.

A cartridge for isolation and purification of a nucleic acid is preferably used to hold the nucleic acid-adsorptive porous membrane(s) in a container provided with at least two openings. When two or more membranes are to be held, they may be the same or different.

When two or more membranes are used, one of the preferable combinations is a porous membrane of an inorganic material and porous membrane of an organic material, e.g., a combination of a glass filter and porous membrane of regenerated cellulose. The other combinations include that of a nucleic acid-adsorptive porous membrane and porous membrane of an organic material showing no adsorption of nucleic acid, e.g., a combination of a glass filter and porous membrane of nylon or polysulfone.

The nucleic acid isolation/purification cartridge holds no member except the nucleic acid-adsorptive porous membrane which it holds in a container provided with at least two openings. The preferable materials for the container include plastics, e.g., polypropylene, polystyrene, polycarbonate and polyvinyl chloride. A biodegradable material is also preferable for the container. The container may be transparent or colored.

The nucleic acid isolation/purification cartridge may be provided with a means for discerning the individual cartridges from each other. These means include a bar code and magnetic tape.

The nucleic acid isolation/purification cartridge may be structured in such a way that the nucleic acid-adsorptive porous membrane held in a container provided with at least two openings can be easily removed.

The nucleic acid isolation/purification cartridge which holds the nucleic acid-adsorptive porous membrane can be used for isolation/purification of nucleic acid by the following steps:
(1a) a step of passing a sample solution containing nucleic acid into a cartridge for isolation and purification of a nucleic acid which holds a nucleic acid-adsorptive porous membrane in a container provided with at least two openings, via one opening (inlet port),
(1b) a step of passing the sample solution containing nucleic acid through the nucleic acid-adsorptive porous membrane under a centrifugal force and discharging the solution from the other opening (discharge port) of the cartridge, to hold the nucleic acid in the membrane by adsorption,
(1c) a step of passing a wash solution into the nucleic acid isolation/purification cartridge via the inlet port,
(1d) a step of passing the wash solution through the nucleic acid-adsorptive porous membrane under a centrifugal force and discharging the solution from the discharge port of the cartridge, to wash the membrane while it is adsorbing the acid,
(1e) a step of passing a elution solution into the nucleic acid isolation/purification cartridge via the inlet port, and
(1f) a step of passing the elution solution through the nucleic acid-adsorptive porous membrane under a centrifugal force to desorb the acid from the membrane and discharge the solution from the cartridge.

In another embodiment, the nucleic acid isolation/purification cartridge which holds the nucleic acid-adsorptive porous membrane can be used for isolation/purification of nucleic acid by the following steps:
(2a) a step of passing a sample solution containing nucleic acid into a cartridge for isolation and purification of a nucleic acid which holds a nucleic acid-adsorptive porous membrane in a container provided with at least two openings, via one opening (inlet port),
(2b) a step of passing the sample solution containing nucleic acid through the nucleic acid-adsorptive porous membrane under reduced pressure generated by a differential pressure generator connected to the discharge port and discharging the solution from the other opening (discharge port) of the cartridge, to hold the nucleic acid in the membrane by adsorption,
(2c) a step of passing a wash solution into the nucleic acid isolation/purification cartridge via the inlet port,
(2d) a step of passing the wash solution through the nucleic acid-adsorptive porous membrane under reduced pressure generated by a differential pressure generator connected to the discharge port and discharging the solution from the discharge port of the cartridge, to wash the membrane while it is adsorbing the acid,
(2e) a step of passing a elution solution into the nucleic acid isolation/purification cartridge via the inlet port, and
(2f) a step of passing the elution solution through the nucleic acid-adsorptive porous membrane under reduced pressure generated by a differential pressure generator connected to the discharge port or under a centrifugal force to desorb the acid from the membrane and discharge the solution from the cartridge.

Next, the washing step is described. Washing the used nucleic acid-adsorptive porous membrane will improve nucleic acid recovery yield and efficiency, leading to reduced requirement for the sample containing nucleic acid. Moreover, automating the washing or recovery step simplifies the system and reduces processing time. The washing step may be carried out once before the recovery step, or preferably 2 or more times when nucleic acid purity is more important. A wash solution is supplied by a tube, pipette, automatic injector or means of equivalent function to a cartridge for isolation and purification of a nucleic acid which holds the nucleic acid-adsorptive porous membrane. When supplied to the nucleic acid isolation/purification cartridge via one opening of the cartridge (inlet port, via which the sample solution containing nucleic acid is injected), a wash solution, can be passed through the nucleic acid-adsorptive porous membrane, and discharged from the other opening (discharge port) of the cartridge, where it is passed through the membrane (1) under a centrifugal force, generated by a centrifuge set to direct the force towards a spent solution container which is provided at the discharge port of the cartridge to hold the spent wash solution, or (2) under reduced pressure generated by a differential pressure generator (e.g., dropper, syringe, vacuum pump or power pipette) connected to the discharge port.

A wash solution may be supplied to and discharged from the nucleic acid isolation/purification cartridge via the same opening. Moreover, it may be supplied to and discharged from the cartridge via an opening which is different from an opening via which the sample solution containing nucleic acid is supplied. However, it is more efficient for washing, and hence more preferable, to supply the solution via one opening of the cartridge and discharge it via the other opening after passing it through the nucleic acid-adsorptive porous membrane.

Quantity of a wash solution is preferably 2 μL/mm² or more in the washing step. Increasing the solution quantity improves the washing effect. However, it is preferably 200 L/mm²or less to prevent efflux of the sample while keeping system workability.

A wash solution is preferably passed through the nucleic acid-adsorptive porous membrane at 2 to 1500 L/second·cm², more preferably 5 to 700 L/second·cm². Decreasing the flow rate, or increasing the contact time, improves the washing effect. However, it is preferable in the above range in consideration of speeding-up the nucleic acid isolation/purification process, which is also important.

A wash solution is preferably kept at 4 to 70° C., more preferably room temperature, in the washing step.

The nucleic acid isolation/purification cartridge may be washed while it is mechanically or ultrasonically vibrated, or under a centrifugal force.

A wash solution for the washing step is normally free of an enzyme, e.g., nuclease. However, it may contain an enzyme capable of degrading a contaminant, e.g., protein. Moreover, it may contain deoxyribonuclease, ribonuclease or the like depending on circumstances. Use of a deoxyribonuclease-containing wash solution will allow selective recovery of RNA from a sample. Similarly, use of a ribonuclease-containing wash solution will allow selective recovery of DNA from a sample.

A wash solution for the washing step preferably contains a water-soluble organic solvent and/or water-soluble salt. It should have a function of washing out an impurity in a sample solution, which is adsorbed on the nucleic acid-adsorptive porous membrane together with nucleic acid. Therefore, it should have a composition which desorbs the impurity from the membrane while keeping nucleic acid adsorbed. A water-soluble organic solvent, e.g., alcohol, in which nucleic acid is sparingly soluble, is suitable for desorbing components other than nucleic acid from the membrane. At the same time, incorporation of a water-soluble salt enhances the effect of adsorbing nucleic acid to improve selective desorption of an unnecessary component.

The water-soluble organic solvents to be contained in a wash solution include methanol, ethanol, isopropanol, n-propanol, butanol and acetone, of which ethanol is more preferable. The solvent is incorporated in a wash solution preferably at 20 to 100% by weight, more preferably 40 to 80% by weight.

On the other hand, the water-soluble salt to be contained in a wash solution is preferably a halide salt, in particular chloride. Moreover, it is preferably monovalent or divalent cationic, in particular an alkali or alkali-earth metallic salt, in particular sodium or potassium salt, the former being more preferable. It is incorporated in a wash solution preferably at 10 mmols/L or more. The upper limit is not limited so long as it is not harmful to impurity solubility. However, it is preferably 1 mol/L or less, more preferably 0.1 mols/L. It is particularly preferable that the water-soluble salt is sodium chloride and contained at 20 mmols/L or more.

A wash water is preferably free of chaotropic substance, to diminish possibility of contamination of the recovery step with such a salt subsequent to the washing step. A chaotropic substance, when present in the recovery step, frequently inhibits an enzymatic reaction, e.g., PCR (polymerase chain reaction). Therefore, it is ideally free of a chaotropic substance in consideration of the subsequent enzymatic reactions or the like. Moreover, a chaotropic substance is frequently corrosive and harmful. Dispensing with a chaotropic substance, therefore, is advantageous also viewed from safety of the operators. The chaotropic substances include chaotropic salts, e.g., guanidine salt described earlier, urea, sodium isocyanate, sodium iodide and potassium iodide.

In a conventional technique, a wash water is highly wettable with a container, e.g., cartridge, in a washing step for nucleic acid isolation/purification process, with the result that it frequently remains in the container to contaminate the subsequent recovery step. This will deteriorate recovered nucleic acid purity or reactivity in the subsequent step. It is therefore essential, when adsorption or desorption of nucleic acid is carried out using a container, e.g., cartridge, to prevent a solution for adsorption or washing, in particular the latter, from remaining in the container so that the subsequent step is not affected by the residual solution.

It is therefore preferable that awash solution has a surface tension of less than 0.035 J/m² to minimize the residual solution in a cartridge, thereby preventing contamination of the subsequent step with the solution. Decreasing surface tension of a solution improves its wettability with a cartridge to hold down the residual solution therein.

Conversely, the residual solution may be held down by keeping solution surface tension at 0.035 J/m² or more, because this enhances water repellency of the cartridge to accelerate formation of the droplets and let them trickle down. An adequate level of surface tension should be selected depending on combination of a nucleic acid-adsorptive porous membrane, elution solution and wash solution.

Use of the nucleic acid-adsorptive porous membrane for the present invention can simplify the washing procedure, because (i) the washing step can be completed by passing the solution through the nucleic acid-adsorptive porous membrane once, (ii) the washing step can be carried out at room temperature, and (iii) a elution solution can be injected into the cartridge immediately after the washing step is completed. One or more of the items (i), (ii) and (iii) can be carried out simultaneously. Conventional techniques frequently need a drying step to swiftly remove an organic solvent contained in a wash solution. On the other hand, the nucleic acid-adsorptive porous membrane for the present invention is a thin film and hence can dispense with this step to simplify the washing procedure.

Conventional nucleic acid isolation/purification techniques frequently involve problems caused by sample contamination with a washing solution scattering and deposited on the container walls during the washing step. This type of contamination can be controlling by devising shape of nucleic acid isolation/purification cartridge which holds a nucleic acid-adsorptive porous membrane in the container provided with two openings and shape of spent solution container.

Next, the nucleic acid recovery step is described. It involves desorption of nucleic acid from the nucleic acid-adsorptive porous membrane.

An elution solution is supplied by a tube, pipette, automatic injector or means of equivalent function to a cartridge for isolation and purification of a nucleic acid which holds the nucleic acid-adsorptive porous membrane. It is supplied to the cartridge via one opening of the cartridge (inlet port, via which the sample solution containing nucleic acid is injected), passed through the nucleic acid-adsorptive porous membrane, and discharged from the other opening (discharge port) of the cartridge, where it is passed through the membrane (1) under a centrifugal force, generated by a centrifuge set to direct the force towards a elution solution container which is provided at the discharge port of the cartridge to hold the elution solution, or (2) under reduced pressure generated by a differential pressure generator (e.g., dropper, syringe, vacuum pump or power pipette) connected to the discharge port.

A elution solution may be supplied to and discharged from the nucleic acid isolation/purification cartridge via the same opening. Moreover, it may be supplied to and discharged from the cartridge via an opening which is different from an opening via which the sample solution containing nucleic acid is supplied. However, it is more efficient for recovering, and hence more preferable, to supply the solution via one opening of the cartridge and discharge it via the other opening after passing it through the nucleic acid-adsorptive porous membrane.

Desorption of nucleic may be carried out after adjusting relative volume of the elution solution to that of the sample solution containing nucleic acid prepared from a sample in a controlled manner. Quantity of the recovered solution containing isolated/purified nucleic acid depends on quantity of the sample used. It is generally in a range from several tens to hundreds μL. However, the range may be extended to a range from 1 μL to several tens mL, when sample quantity is very small or a large quantity of nucleic acid is to be isolated/purified.

The elution solution is preferably refined distilled water or aqueous buffer solution, e.g., Tris/EDTA buffer. When recovered nucleic acid is to be subjected to PCR (polymerase chain reaction), a buffer solution for the reaction (e.g., aqueous solution of KCl, Tris-Cl or MgCl2 adjusted to have a final concentration of 50, 10 or 1.5 mmols/L, respectively) may be also used.

A elution solution is preferably kept at a pH level of 2 to 11, more preferably 5 to 9. Its ion strength and salt concentration have significant effects on elution of adsorbed nucleic acid. It preferably has an ion strength of 290 mmols/L or less, and salt concentration of 90 mmols/L or less. The elution solution satisfying these conditions can improve recovery rate of nucleic acid and increase its yield.

Decreasing volume of a elution solution relative to that of an initial sample solution containing nucleic acid can increase concentration of nucleic acid in the recovered solution. The elution solution/sample solution ratio is preferably in a range from 1/100 to 99/100 by volume, more preferably 1/10 to 9/10. This ratio allows nucleic acid to be concentrated in a simple manner in the nucleic acid isolation/purification step without needing an additional concentration step. These steps can yield a solution containing nucleic acid at a higher concentration than the sample.

Another method desorbs nucleic acid using a larger quantity of elution solution than an initial sample solution containing nucleic acid. This will yield a recovered solution containing nucleic acid at a desired concentration, e.g., concentration suitable for a subsequent step, e.g., that for PCR. The elution solution/sample solution ratio is preferably in a range from 1/1 to 50/1 by volume, more preferably 1/1 to 5/1. This brings a merit of eliminating a time-consuming step of adjusting the recovered solution discharged from the nucleic acid isolation/purification step. Moreover, use of a sufficient quantity of elution solution can increase rate of recovering nucleic acid from the porous membrane.

Nucleic acid can be simply recovered by changing elution solution temperature for a specific purpose. For example, desorption of nucleic acid from a porous membrane while keeping a elution solution at 0 to 10° C. can produce a nucleic acid solution simply and efficiently while controlling actions of nuclease to prevent from degradation of a nucleic acid without using any reagent or procedure for enzyme-aided degradation.

Moreover, nucleic acid can be recovered at generally adopted room temperature by keeping a elution solution at 10 to 35° C., and desorbed without needing a sophisticated process for its isolation/purification.

Another procedure keeps a elution solution at higher temperature, e.g., 35 to 70° C. This can recover nucleic acid from a nucleic acid-adsorptive porous membrane simply and in a high yield without needing a sophisticated nucleic acid desorption procedure.

Number of elution solution injection is not limited. It may be injected once or 2 or more times. It is normally injected once when nucleic acid is to be isolated and purified swiftly and simply. However, it may be injected 2 or more times, when a large quantity of nucleic acid is to be recovered.

An elution solution can be used for the recovery step in such a way to keep its composition useful for a subsequent step. For example,. isolated/purified nucleic acid is frequently amplified by PCR (polymerase chain reaction), and the isolated/purified nucleic acid solution can be diluted with the above-described buffer solution suitable for PCR. Use of such a buffer solution is preferable, because the elution solution can be passed to the PCR step simply and swiftly.

A nucleic acid elution solution for the recovery step may be incorporated with a stabilizer to prevent degradation of the recovered nucleic acid. The stabilizers useful for the present invention include antibacterial agents, antifungal agents and nucleic acid degradation inhibitors. EDTA is one of nuclease inhibitors. In another embodiment, a stabilizer may be added beforehand to the elution solution container.

The elution solution container for the recovery step is not limited. For example, it may be made of a material which shows no absorbance at 260 nm. A nucleic acid solution held in such a container can be analyzed directly for concentration without being transferred to another container. These materials include, but not limited to, quartz glass.

It is preferable to automate the nucleic acid isolation/purification step which treats a sample containing nucleic acid using a cartridge for isolation and purification of a nucleic acid holding the nucleic acid-adsorptive porous membrane in a container provided with at least two openings, and centrifuge and/or differential pressure generator. This can simplify and speedup the isolation/purification step, and, at the same time, produce nucleic acid of stable quality irrespective of skill of the operator.

### EXAMPLES

The present invention is described in more detail by EXAMPLES, which by no means limit the present invention.

(1) Preparation of the Nucleic Acid Isolation/Purification Cartridge

The nucleic acid isolation/purification cartridge having a container (inner diameter: 7 mm) was prepared by setting a nucleic acid-adsorptive porous membrane in a dedicated space in the container.

The nucleic acid-adsorptive porous membrane (thickness: 70 μm, average pore size: 5.0 μm) was prepared by two-dimensionally spreading a solution comprising 100 parts by weight of an acetyl cellulose mixture (triacetyl cellulose/diacetyl cellulose: 6/4 by weight) dissolved in 250 parts by weight of a mixed organic solvent (dichloromethane/methanol: 8/2 by weight) and then removing the mixed solvent by evaporation. The membrane was immersed in a 2 mols/L aqueous solution of sodium hydroxide for 20 minutes for saponification (saponification degree: about 100%), and then set in the cartridge.

The saponification treatment reduced the average membrane pore size from 5.0 μm to 2.5 μm.

(2) Preparation of the nucleic acid solubilizing reagent and wash solution The following nucleic acid solubilizing reagent, wash solution and elution solution compositions were prepared.

| | | |
|---|---|---|
| (Nucleic acid solubilizing reagent) | 382 | g |
| Guanidine hydrochloride (Life Technology) | 12.1 | g |
| Tris (Life Technology) | 10 | g |
| TrintonX-100 (ICN) | | |
| Distilled water (ph 7.0) | 1000 | mL |
| (Wash solution) | | |
| NaCl | 100 | mmols/L |
| Tris-HCl | 10 | mmols/L |
| Ethanol (60% by volume) | 1000 | mL |

### Example 1

(3-1) Nucleic Acid Isolation/Purification Procedure

A mixture of 200 μL of human whole blood sample, 200 μL of a nucleic acid solubilizing reagent and 20 μL of a 20 mg/mL (200 Units/mL) solution of protease (Proteinase, Bacterial TypeXXIV, SIGMA) was incubated at 60° C. for 10 minutes. The incubated mixture was stirred together with 200 μL of ethanol to prepare the sample solution containing nucleic acid. This sample solution was injected into the nucleic acid isolation/purification cartridge, prepared in the (1) described above, holding the nucleic acid-adsorptive porous membrane of a mixture of saponified acetyl cellulose compounds of different acetyl value, via one opening (inlet port) ; passed through the membrane under a centrifugal force for 1 minute, generated by a centrifuge (MX-150, TOMY) operating at 8000 rpm, set to direct the force towards a spent solution container provided in the cartridge to hold the spent sample solution, to bring the sample solution into contact with the membrane; and discharged via the other opening (discharge port) of the cartridge. Next, the cartridge was removed from the centrifuge, and the spent solution container was replaced by another one. Then, 500 μL of the wash solution (described in Table 1) was injected into the cartridge via the inlet port; passed through the membrane under a centrifugal force for 1 minute, generated by a centrifuge (MX-150, TOMY) operating at 8000 rpm, set to direct the force towards the spent solution container provided in the cartridge; and discharged via the discharge port. Next, the cartridge was removed from the centrifuge, and the spent solution container was replaced by a elution solution container. Then, 200 μL of a elution solution (sterilized water, pH: of 7.0) was injected into the cartridge via the inlet port; passed through the membrane under a centrifugal force for 1 minute, generated by a centrifuge (MX-150, TOMY) operating at 8000 rpm, set to direct the force towards the elution solution container provided in the cartridge; and discharged via the discharge port to recover the solution.

### Comparative Example 1

The nucleic acid isolation/purification was carried out in COMPARATIVE EXAMPLE 1 in the same manner as in EXAMPLE 1, except that the nucleic acid-adsorptive porous membrane was replaced by a glass filter (silica gel filter, film thickness: 1000 μm).

(4-1) Confirmation of Nucleic Acid Isolation/Purification

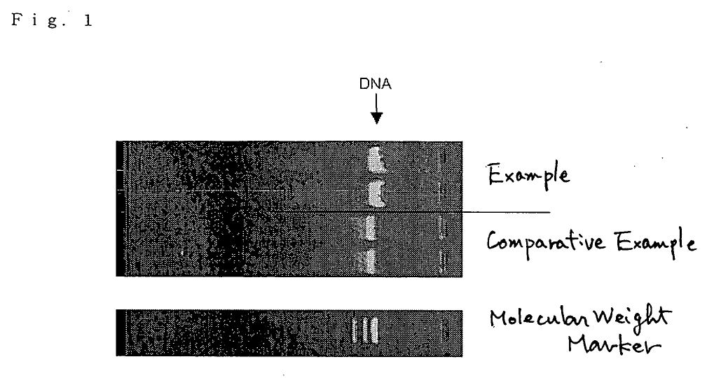

Electrophoresis with agarose gel (0.5% agarose containing ethidium bromide, 100 V, 30 minutes) was carried out for each of the solutions recovered in EXAMPLE 1. and COMPARATIVE EXAMPLE 1, where λHindIII digest (Gibco) was used as a molecular weight marker. The results are given in FIG. 1.

Each of the solutions recovered in EXAMPLE 1 and COMPARATIVE EXAMPLE 1 was analyzed for absorbance at 260 nm to estimate yield of isolated/purified DNA. The yields of the DNA recovered in EXAMPLE 1 and COMPARATIVE EXAMPLE 1 were 8.3 and 8.8 μg, and 5.9 and 6.2 μg, respectively.

It is found, based on the results shown in FIG. 1 and high DNA yield determined by absorbance at 260 nm, that nucleic acid can be isolated/purified in high yield by passing a sample solution containing nucleic acid under a centrifugal force through a nucleic acid-adsorptive porous membrane of a mixture of saponified acetyl cellulose compounds of different acetyl value, held in a cartridge for isolation and purification of a nucleic acid.

### Example 2

(3-2) Nucleic Acid Isolation/Purification Procedure

A mixture of 200 μL of human whole blood sample, 20μL of a nucleic acid solubilizing reagent and 20 μL of a 20 mg/mL (200 Units/mL) solution of protease (Bacterial TypeXXIV, SIGMA) was incubated at 60° C. for 10 minutes. The incubated mixture was stirred together with 200 μL of ethanol to prepare the sample solution containing nucleic acid. This sample solution was injected into the nucleic acid isolation/purification cartridge, prepared in the (1) described above, holding the nucleic acid-adsorptive porous membrane of a mixture of saponified acetyl cellulose compounds of different acetyl value, via one opening (inlet port); passed through the membrane under reduced pressure (−50 kPa) generated by a differential pressure generator (vacuum pump) connected to the other opening (discharge port) of the cartridge, to bring the sample solution into contact with the membrane; and discharged via the discharge port. Next, 500 μL of the wash solution prepared in the (1) described above was injected into the cartridge via the inlet port; passed through the membrane under reduced pressure (−50 kPa) generated by a differential pressure generator connected to the other opening (discharge port) of the cartridge; and discharged via the discharge port. Then, 200 μL of a elution solution (sterilized water, pH: of 7.0) was injected into the cartridge via the inlet port; passed through the membrane under reduced pressure (−50 kPa) generated by a differential pressure generator connected to the discharge port of the cartridge, and discharged via the discharge port to recover the solution.

### Comparative Example 2

The nucleic acid isolation/purification was carried out in COMPARATIVE EXAMPLE 2 in the same manner as in EXAMPLE 2, except that the nucleic acid-adsorptive porous membrane was replaced by a glass filter (silica gel filter, film thickness:

(4-b) Confirmation of Nucleic Acid Isolation/Purification

Each of the solutions recovered in EXAMPLE 2 and COMPARATIVE EXAMPLE 2 was analyzed for absorbance at 260 nm to estimate yield of isolated/purified DNA. The yields of the DNA recovered in EXAMPLE 2 and COMPARATIVE EXAMPLE 2 were 8.3 and 8.8 μg, and 5.9 and 6.2 μg, respectively.

It is found, based on the high DNA yield determined by absorbance at 260 nm, that nucleic acid can be isolated/purified in high yield by passing a sample solution containing nucleic acid under reduced pressure through a nucleic acid-adsorptive porous membrane of a mixture of saponified acetyl cellulose compounds of different acetyl value, held in a cartridge for isolation and purification of a nucleic acid.

This application is based on Japanese patent applications JP 2003-373024, filed on Oct. 31, 2003, JP 2003-373111, filed on Oct. 31, 2003, and JP 2004-277933, filed on Sep. 24, 2004, the entire content of which is hereby incorporated by reference, the same as if set forth at length.

1. A method of isolating and purifying a nucleic acid, comprising the steps of:
(1a) passing a sample solution containing a nucleic acid through a nucleic acid-adsorptive porous membrane to adsorb the nucleic acid to the nucleic acid-adsorptive porous membrane;
(2a) passing a wash solution through the nucleic acid-adsorptive porous membrane to wash the nucleic acid-adsorptive porous membrane while adsorbing the nucleic acid; and
(3a) passing a elution solution through the nucleic acid-adsorptive porous membrane to desorb the nucleic acid from the nucleic acid-adsorptive porous membrane,
wherein each of the sample solution, the wash solution and the elution solution in each of the steps (1a), (2a) and (3a) is passed through the nucleic acid-adsorptive porous membrane under a centrifugal force.

2. The method of isolating and purifying a nucleic acid according to claim 1, which comprising using a cartridge for isolation and purification of a nucleic acid, the cartridge comprising: a container provided with at least two openings; and the nucleic acid-adsorptive porous membrane provided in the container,
wherein each of the sample solution, the wash solution and the elution solution in each of the steps (1a), (2a) and (3a) is injected into the cartridge via one of the at least two openings of the container, is passed through the nucleic acid-adsorptive porous membrane by centrifuging the cartridge, and is discharged from the other opening of the container.

3. A method of isolating and purifying a nucleic acid, comprising the steps of:
(1b) passing a sample solution containing a nucleic acid through a nucleic acid-adsorptive porous membrane to adsorb the nucleic acid to the nucleic acid-adsorptive porous membrane;
(2b) passing a wash solution through the nucleic acid-adsorptive porous membrane to wash the nucleic acid-adsorptive porous membrane while adsorbing the nucleic acid; and
(3b) passing a elution solution through the nucleic acid-adsorptive porous membrane to desorb the nucleic acid from the nucleic acid-adsorptive porous membrane,
wherein each of the sample solution and the wash solution in each of the step (1b) or (2b) is passed through the nucleic acid-adsorptive porous membrane under reduced pressure, and the elution solution in the step (3b) is passed through the nucleic acid-adsorptive porous membrane under reduced pressure or centrifugal force.

4. The method of isolating and purifying a nucleic acid according to claim 3, which comprising using a cartridge for isolation and purification of a nucleic acid, the cartridge comprising: a container provided with at least two openings; and the nucleic acid-adsorptive porous membrane provided in the container,
wherein each of the sample solution and the wash solution in each of the step (1b) or (2b) is injected into the cartridge via one of the at least two of openings of the container, is passed through the cartridge under reduced pressure generated by a differential pressure generator connected to the other opening of the container, and is discharged from the other opening of the container, and
the elution solution in the step (3b) is injected into the cartridge via one of the at least two of openings of the container, is passed through the cartridge under reduced pressure generated by a differential pressure generator connected to the other opening of the container or under a centrifugal force, and is discharged from the other opening of the container.

5. The method of isolating and purifying a nucleic acid according to claim 2, wherein the nucleic acid-adsorptive porous membrane is a porous membrane capable of adsorbing a nucleic acid by an interaction involving substantially no ionic bond.

6. The method of isolating and purifying a nucleic acid according to claim 5, wherein the porous nucleic acid-adsorptive membrane is a porous membrane comprising an organic polymer having a polysaccharide structure.

7. The method of isolating and purifying a nucleic acid according to claim 6, wherein the porous membrane comprising an organic polymer having a polysaccharide structure is a porous membrane comprising a mixture of acetyl cellulose having a different acetyl value.

8. The method of isolating and purifying a nucleic acid according to claim 7, wherein the mixture of acetyl cellulose having a different acetyl value is a mixture of triacetyl cellulose and diacetyl cellulose.

9. The method of isolating and purifying a nucleic acid according to claim 8, wherein the mixture contains triacetyl cellulose and diacetyl cellulose in a ratio of 99/1 to 1/99 by weight.

10. The method of isolating and purifying a nucleic acid according to claim 7, wherein the mixture of acetyl cellulose having a different acetyl value is a mixture of triacetyl cellulose and monoacetyl cellulose.

11. The method of isolating and purifying a nucleic acid according to claim 7, wherein the mixture of acetyl cellulose having a different acetyl value is a mixture of triacetyl cellulose, diacetyl cellulose and monoacetyl cellulose.

12. The method of isolating and purifying a nucleic acid according to claim 7, wherein the mixture of acetyl cellulose having a different acetyl value is a mixture of diacetyl cellulose and monoacetyl cellulose.

13. The method of isolating and purifying a nucleic acid according to claim 6, wherein the porous membrane comprising an organic polymer having a polysaccharide structure is a porous membrane comprising an organic material containing saponified acetyl cellulose.

14. The method of isolating and purifying a nucleic acid according to claim 13, wherein a saponification degree of the saponified acetyl cellulose is 5% or more.

15. The method of isolating and purifying a nucleic acid according to claim 13, wherein the porous membrane comprising an organic material containing saponified acetyl cellulose is a porous membrane comprising an organic material containing a saponified mixture of acetyl cellulose having a different acetyl value.

16. The method of isolating and purifying a nucleic acid according to claim 15, wherein a saponification degree of the saponified mixture of acetyl cellulose having a different acetyl value is 5% or more.

17. The method of isolating and purifying a nucleic acid according to claim 16, wherein the organic material containing the saponified mixture of acetyl cellulose having a different acetyl value is a saponified mixture of triacetyl cellulose and diacetyl cellulose.

18. The method of isolating and purifying a nucleic acid according to claim 17, wherein the ratio of triacetyl cellulose and diacetyl cellulose in the saponified mixture is 99/1 to 1/99 by weight.

19. The method of isolating and purifying a nucleic acid according to claim 16, wherein the organic material containing the saponified mixture of acetyl cellulose having a different acetyl value is a saponified mixture of triacetyl cellulose and monoacetyl cellulose.

20. The method of isolating and purifying a nucleic acid according to claim 16, wherein the organic material containing the saponified mixture of acetyl cellulose having a different acetyl value is a saponified mixture of triacetyl cellulose, diacetyl cellulose and monoacetyl cellulose.

21. The method of isolating and purifying a nucleic acid according to claim 16, wherein the organic material containing the saponified mixture of acetyl cellulose having a different acetyl value is a saponified mixture of diacetyl cellulose and monoacetyl cellulose.

22. The method of isolating and purifying a nucleic acid according to claim 13, wherein the nucleic acid-adsorptive porous membrane after saponification treatment has an average pore size smaller than that before the saponification treatment.

23. The method of isolating and purifying a nucleic acid according to claim 22, wherein the raito of an average pore size of the nucleic acid-adsorptive porous membrane after saponification treatment to that before the saponification treatment is 0.8 or less.

24. The method of isolating and purifying a nucleic acid according to claim 6, wherein the nucleic acid-adsorptive porous membrane is a porous membrane containing a regenerated cellulose.

25. The method of isolating and purifying a nucleic acid according to claim 5, wherein the nucleic acid-adsorptive porous membrane is a porous membrane obtained by treating a porous membrane comprising an organic material free of a hydrophilic group to introduce a hydrophilic group.

26. The method of isolating and purifying a nucleic acid according to claim 25, wherein the introduction of hydrophilic group to the porous membrane is carried out by binding a graft polymer chain having a hydrophilic group to the porous membrane.

27. The method of isolating and purifying a nucleic acid according to claim 5, wherein the nucleic acid-adsorptive porous membrane is a porous membrane obtained by coating an organic material free of a hydrophilic group with a material having a hydrophilic group to introduce a hydrophilic group.

28. The method of isolating and purifying a nucleic acid according to claim 27, wherein the material having a hydrophilic group is an organic polymer having a hydrophilic group.

29. The method of isolating and purifying a nucleic acid according to claim 5, wherein the nucleic acid-adsorptive porous membrane is a porous membrane comprising an inorganic material having a hydrophilic group in itself.

30. The method of isolating and purifying a nucleic acid according to claim 5, wherein the nucleic acid-adsorptive porous membrane is a porous membrane obtained by treating a porous membrane comprising an inorganic material free of a hydrophilic group to introduce a hydrophilic group.

31. The method of isolating and purifying a nucleic acid according to claim 30, wherein the introduction of a hydrophilic group to the porous membrane is carried out by binding a graft polymer chain having a hydrophilic group to the porous membrane.

32. The method of isolating and purifying a nucleic acid according to claim 5, wherein the nucleic acid-adsorptive porous membrane is a porous membrane obtained by coating a porous membrane comprising an inorganic material free of hydrophilic group with a material having a hydrophilic group to introduce a hydrophilic group.

33. The method of isolating and purifying a nucleic acid according to claim 32, wherein the material having a hydrophilic group is an organic polymer having a hydrophilic group.

34. The method of isolating and purifying a nucleic acid according to any one of claims 25, 27, 29, 30 and 32, wherein the hydrophilic group is a hydroxyl group.

35. The method of isolating and purifying a nucleic acid according to claim 5, wherein the nucleic acid-adsorptive porous membrane is a porous membrane, in which the front and back sides of the porous membrane are asymmetrical.

36. A cartridge for isolation and purification of a nucleic acid, comprising a nucleic acid-adsorptive porous membrane and a container provided with at least two openings, the cartridge being used for carrying out the method of isolating and purifying a nucleic acid according to claim 5.

37. A kit comprising: a cartridge for isolation and purification of a nucleic acid; and a reagent, the kit being used for carrying out the method of isolating and purifying a nucleic acid according to claim 5.
